(19) United States
(12) Patent Application Publication
(10) Pub. No.: US 2025/0017465 A1
(43) Pub. Date: Jan. 16, 2025

(54) ASSESSING EYE FLOATERS

(71) Applicant: Alcon Inc., Fribourg (CH)

(72) Inventor: Steven T. Charles, Memphis, TN (US)

(21) Appl. No.: 18/766,927

(22) Filed: Jul. 9, 2024

Related U.S. Application Data
(60) Provisional application No. 63/512,845, filed on Jul. 10, 2023.

(51) Int. Cl. A61B 3/15; A61B 3/00; A61B 3/113

(52) U.S. Cl. CPC A61B 3/152; A61B 3/0033; A61B 3/113

(57) ABSTRACT

In certain embodiments, a system for assessing a floater in the eye of a patient includes a display device, input device, and computer. The display device displays a target and background. The background has an alignment point and alignment axis. The eye axis of the eye is substantially aligned with the alignment axis when the eye is fixating on the alignment point. The input device receives patient input from the patient. The computer provides target display input to the display device to yield the target moving according to a pattern for the eye to follow. The eye axis moves relative to the alignment axis as the eye follows the moving target. The computer records patient input indicating that the patient sees the floater. The computer calculates a severity score, which indicates the severity of the floater, according to the patient input and generates a report that describes the severity score.

### TECHNICAL FIELD

The present disclosure relates generally to ophthalmic systems, and more particularly to an ophthalmic system for assessing eye floaters.

### BACKGROUND

Eye floaters are tiny strands of vitreous or other tissue in the eye that stick together and cast shadows on the retina. The shadows appear as small dark shapes that float across your vision. People differ in their reactions to a floater. Some people barely even notice a floater, while other people may be distressed by a floater. The difference in reactions may be due to a combination of the floater and the psychophysical perceptions of the patient.

Known methods for assessing floaters are flawed. For example, an eye doctor can check for the presence of floaters as part of an eye exam. However, merely determining the presence of a floater does not take into account the perception of the patient. As another example, the patient can describe the floater. However, such descriptions are typically not precise and may exaggerate the severity of the floater, which may lead to the unnecessary removal of an otherwise benign floater. Moreover, an insurance company might consider these subjective descriptions as unreliable and thus not strong evidence of the severity of the floater.

### BRIEF SUMMARY

In certain embodiments, a system for assessing a floater in the eye of a patient includes a display device, input device, and computer. The display device displays a target and background. The background has an alignment point and alignment axis. The eye axis of the eye is substantially aligned with the alignment axis when the eye is fixating on the alignment point. The input device receives patient input from the patient. The computer provides target display input to the display device to yield the target moving relative to the background according to a pattern for the eye to follow. The eye axis moves relative to the alignment axis as the eye follows the moving target. The computer receives and records patient input indicating that the patient sees the floater. The computer calculates a severity score, which indicates the severity of the floater, according to the patient input and generates a report that describes the severity score.

Embodiments may include none, one, some, or all of the following features:
The computer provides the display input to: move the target to a first position; maintain the target at the first position for a dwell period within a range of 1 to 2 seconds; and then move the target to a second position.
The computer provides the display input to move the target to a position that yields a fixation direction relative to the alignment axis of the background, where a fixation angle between the fixation direction and alignment axis is within a range of 10 to 30 degrees.
The computer provides the display input to perform the following for one or more iterations: move the target in a first direction; move the target to the alignment point; move the target in a second direction different from the first direction; and move the target to the alignment point.
The computer provides the display input to adjust visual contrast between the target and the background in response to a request.
The computer calculates the severity score by determining the location of the floater according to the patient input and determining a location score according to the location of the floater. The computer then calculates the severity score in accordance with the location score, where a location closer to the eye axis corresponds to a greater severity and a location farther away from the eye axis corresponds to a lesser severity.
The computer calculates the severity score by determining a density score indicating the density of the floater and calculating the severity score in accordance with the density score.
The computer provides the display input to adjust intensity of the background and records the patient input in response to the adjusted intensity. The computer then determines a density score according to the recorded patient input, the density score indicating the density of the floater.
The computer provides the display input to adjust contrast between the target and background and records the patient input in response to the adjusted contrast. The computer then determines a density score according to the recorded patient input, the density score indicating the density of the floater.
The computer calculates the severity score by determining an occurrence score in response to the number of times the patient indicates that the patient sees the floater. The computer then calculates the severity score in accordance with the occurrence score, where a greater number of times corresponds to a greater severity and a lesser number of times corresponds to a lesser severity.
The computer calculates the severity score by detecting a reaction time between moving the target and receiving patient input provided when a floater is detected. The computer determines an occurrence score in according to the reaction time and then calculates the severity score in accordance with the occurrence score, where a shorter reaction time corresponds to a greater severity and a longer reaction time corresponds to a lesser severity.
The computer calculates the severity score by determining a situational score that indicates factors relevant to a patient situation. The computer calculates the severity score in accordance with the situational score.
The computer calculates the severity score by receiving a patient description describing the floater and calculating the severity score in accordance with the patient description.
The computer provides test instruction display input to the display device to yield test instructions that include an instruction to provide patient input to the input device when a floater is detected.
The computer provides calibration display input to the display device to yield calibration instructions that include an instruction to provide patient input to the input device when a simulated floater is detected.
The computer provides calibration display input to the display device to display a simulated floater and receives patient input indicating that the patient sees the simulated floater. The computer then determines a reaction time between displaying the simulated floater and receiving the patient input and adjusts a dwell period of the pattern according to the reaction time.
The computer provides calibration display input to the display device to yield calibration instructions to display simulated floaters having floater characteristics. The computer receives patient input indicating that the patient sees one or more simulated floaters and identifies the characteristics of the simulated floaters indicated by the patient input.
The computer provides trial run display input to the display device to yield trial run instructions for conducting a trial run of a test.
The system includes an eye tracker that tracks movement of the eye to determine the fixation position of the eye.
The target or the background is lighter than the other of the target or the background.
The target has a first color, and the background has a second color that is a complement of the first color.
The input device has an actuator that receives movement input from the patient.
The input device has a microphone that detects sound input from the patient.
The input device has a motion detector that detects gesture input from the patient.

### BRIEF DESCRIPTION OF THE DRAWINGS

FIG. 1 illustrates an example of a floater assessment system, according to certain embodiments;

FIGS. 2 through 4B illustrate examples of fixation targets and a background that may be displayed to a patient by the system of FIG. 1, according to certain embodiments: FIG. 2 shows a target moving relative to the background according to an example target pattern, FIG. 3 shows a side view of an eye viewing a target displayed by a display device, and FIGS. 4A and 4B show examples of fixation targets;

FIG. 5 illustrates an example of an assessment application that may be used in the system of FIG. 1, according to certain embodiments;

FIGS. 6A to 6C illustrate examples of graphical user interfaces (GUIs) that the system of FIG. 1 displays to provide instructions to a patient, according to certain embodiments;

FIGS. 7A and 7B illustrate examples of the packaging of the system of FIG. 1, according to certain embodiments; and

FIG. 8 illustrates an example of a method for assessing a floater in an eye of a patient that may be performed by the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

In certain embodiments, a system assesses the severity of an eye floater by having a patient fixate on a moving target and then provide input (e.g., click a button) when the patient sees a floater. For example, the target starts near the outer part of a display and then moves towards the center of the display. The patient may see the floater while looking straight ahead at the center and then click the button. The system assesses the floater in response to the patient input. For example, the system may determine the location of the floater within the eye according to where the eye was fixating when input was received. From the assessment, the system calculates a severity score that indicates the severity of the floater.

Certain embodiments of the system offer advantages. For example, the system calculates the severity score in a reproducible manner that takes into account the perception of the patient. Since the calculation is reproducible, third parties such as insurance companies may regard the assessment as strong evidence of a floater that requires treatment. As another example, the system may present instructions for taking the floater assessment test, such that the patient does not require assistance to take the test. Thus, the assessment does not require the time of an eye doctor. As yet another example, the system may be packaged in a convenient compact housing, such as in a desktop device or wearable goggles.

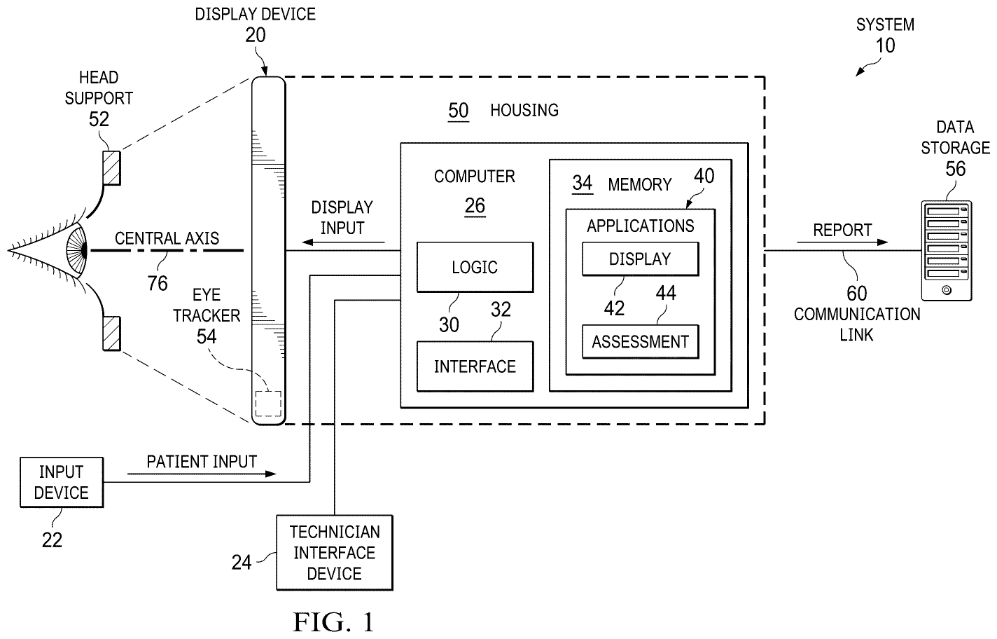

FIG. 1 illustrates an example of a floater assessment system 10, according to certain embodiments. As an overview of the example, system 10 includes a display device 20, a patient input device 22, a technician interface (IF) device 24, a computer 26 (which includes logic 30, an interface 32, and memory 34 that stores applications 40 such as display 42 and assessment 44 applications), a housing 50, a head support 52, and an eye tracker 54 (in certain embodiments). System 10 communicates with data storage 56 via a communication link 60.

In an example of operation, system 10 instructs the patient to follow a moving fixation target and provide input into patient input device 22 when the patient sees a floater. Computer 26 instructs display device 20 to move the fixation target relative to the background (e.g., move the target from an outer part of the background towards the center) for the eye to follow. Computer 20 receives input from the patient indicating that the patient sees the floater (e.g., while looking straight ahead at the center). Computer 20 calculates a severity score that indicates severity of the floater and generates a report that includes the severity score.

Turning to example components of system 10, display device 20 displays an image generated from image data received as display input from computer 26. Display device 20 is any suitable device that can generate and display an image, such as a computer screen or monitor, e.g., an LCD, LED, or other video screen.

Patient input device 22 receives input from a patient and communicates the input to computer 26. Patient input device 22 may be any suitable device that can receive any suitable human input, e.g., a movement or a sound. For example, input device 22 may include an actuator (such as a button, lever, knob, key, or other physical component) that a human can move (e.g., depress, turn, or switch) to provide input. As another example, input device 22 may include a microphone that can detect a sound (e.g., speech) made by a human. As yet another example, input device 22 may include a motion detector that can detect motion (e.g., a gesture such as a hand wave) made by a human.

Technician interface (IF) device 24 receives input from a user conducting the test (such as a medical technician) and provides output to the user. The input may be received via, e.g., a keyboard, actuator, or touchscreen, and output may be provided via, e.g., a display device such as a computer screen or monitor. In certain embodiments, technician IF device 24 includes mainly starting and stopping controls that start and stop, respectively, the floater assessment procedure.

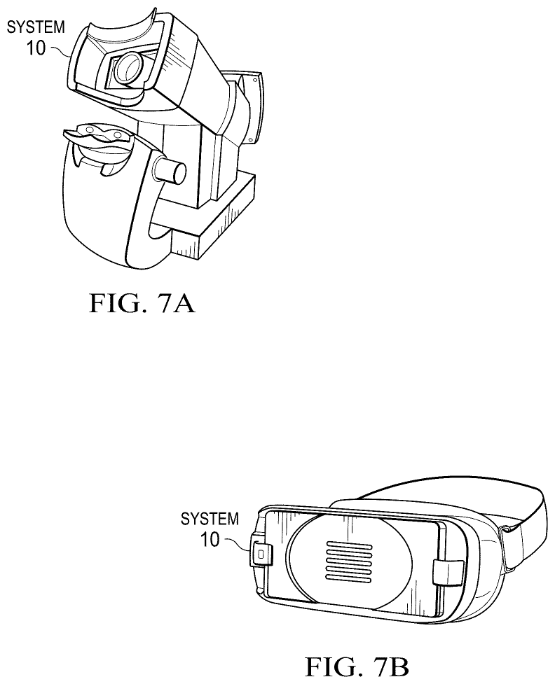

Housing 50 contains and/or supports one or more components of system 10, e.g., display device 20, patient input device 22, technician interface (IF) device 24, computer 26, head support 52, and/or eye tracker 54. Housing 50 may have any suitable shape or size, e.g., a desktop device or wearable goggles. In the illustrated example, housing 50 contains display device 20, computer 26, and eye tracker 54 and provides support for head support 52. In the example, a patient places their head in contact with head support 52 to view display device 20. In this position, the patient eye may be in the proper location and orientation relative to display device 20 to see the target and background. Other examples of housing 50 are described in FIGS. 7A and 7B.

In certain embodiments, eye tracker 54 tracks movement of the eye to determine the fixation position of the eye. The position of the eye may be tracked by tracking a feature (e.g., pupil, iris, or limbus) of the eye. Computer 26 can use the position of the eye to determine where the eye is fixating in order to validate that the eye is fixating on the target.

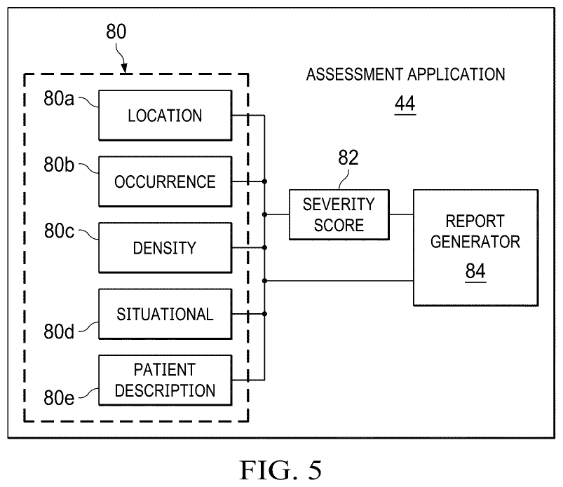

Computer 26 controls the components of system 10, e.g., display device 20, patient input device 22a technician interface (IF) device 24, and/or eye tracker 54. In the illustrated example, computer 26 uses display application 42 to generate display input that instructs display device 20, e.g., to display the target moving relative to the background for the eye to follow or to display instructions or other information. Computer 26 receives the input from the patient that the patient sees the floater and uses assessment application 44 to calculate a severity score that indicates severity of the floater, or how much the floater disrupts vision. Computer 26 is described in more detail with reference to FIG. 5.

Data storage 56 receives reports generated by computer 26, and may comprise any suitable data storage memory. Communication link 60 communicates information between computer 26 and data storage 56. Communication link 60 may comprise any suitable wired and/or wireless link that can communicate computer data. For example, communication link 60 may be compatible with an electronic medical records standard such as the Digital Imaging and Communications in Medicine (DICOM) Standard.

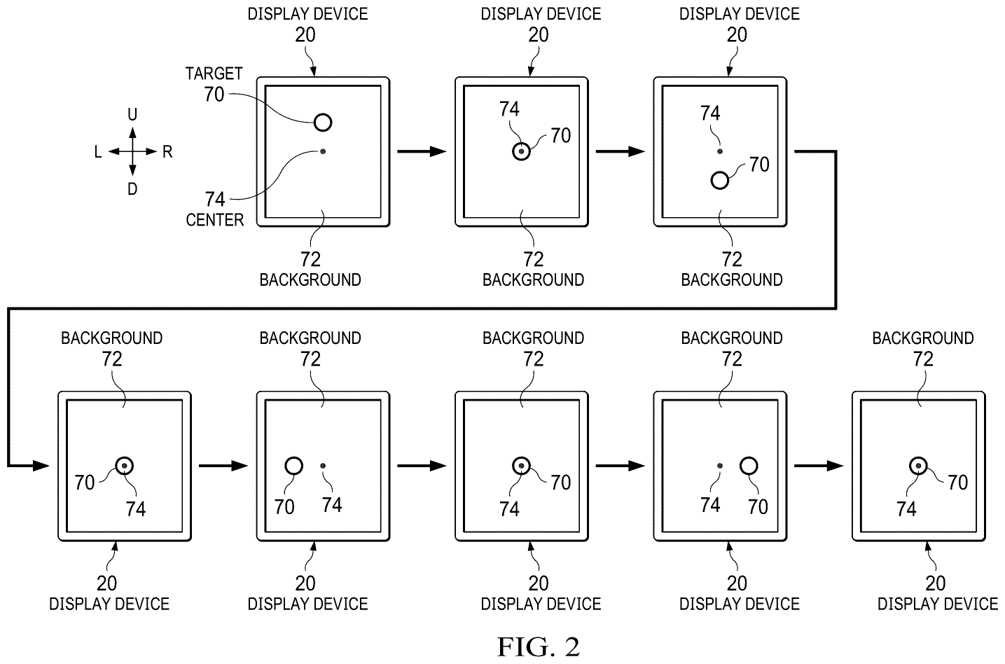

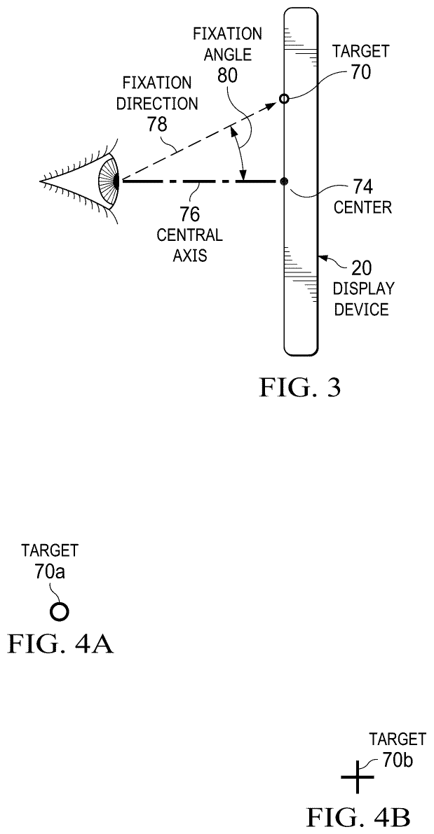

FIGS. 2 through 4 illustrate examples of fixation targets 70 and a background 72 that may be displayed to a patient by system 10, according to certain embodiments. FIG. 2 shows target 70 moving relative to background 72 according to an example target pattern. FIG. 3 shows a side view of an eye viewing target 70 displayed by display device 20. FIGS. 4A and 4B show examples of fixation targets 70.

FIG. 2 shows target 70 moving relative to background 72 according to an example target pattern. For ease of explanation, FIG. 2 makes reference to Up (U), Down (D), Left (L), and Right (R) directions. In the example, target 70 is moved up to an upper point, then to center 74, down to a lower point, back to center 74, left to a left point, back to center 74, right to a right point, and back to center 74. During the test procedure, the patient's fixation repeatedly changes (using saccadic eye motion) from straight ahead, to an angular position relative to straight ahead, back to straight ahead, to another angular position, etc. Floaters slowly drift around after the deviated fixation positions, so the procedure can determine how frequently a floater interferes with central fixation.

The target pattern may include any suitable variation. As an example of a variation, the path of target 70 may be continuous or discontinuous or may include continuous and discontinuous portions. Target 70 may move along a continuous path that connects points A and B of the path (e.g., pursuit motion) or may move along a discontinuous path such that target 70 appears point A and then at point B, but does not appear between points A and B. For example, target 70 may move continuously from the upper point to center 74 by appearing at points between the upper point and center 74, or target 70 may move discontinuously from the upper point to center 74 by appearing at the upper point, disappearing from the upper point, and appearing at center 74.

As another example of a variation, a pattern may move target 70 to more or fewer outer points. The pattern may move target 74 in any direction between the U, D L, or R directions, e.g., a direction or directions that bisects or trisects the angle between the U, D L, and/or R directions.

Target 70 may have any suitable size, shape, intensity, or color (expressed as, e.g., greyscale or spectral color). In FIGS. 4A and 4B, target 70a is a circle, and target 70b is a cross. Background 72 may have any suitable intensity or color. The contrast between target 70 and background 72 should be sufficiently high to make target 70 visible relative to background 72. For example, target 70 may be darker than background 72 (such as a black target 70 and a white background 72) or target 70 may be lighter than background 72 (such as a white target 70 and a black background 72). As another example, target 70 may be a color that is the complement of that of the background 72.

In certain embodiments, computer 26 may change the intensity and/or color of target 70 and/or background 72 during the floater assessment procedure. For example, the contrast between target 70 and background 72 may be adjusted in response to a patient request to improve floater visualization. As another example, the intensity of background 72 may be adjusted to, e.g., estimate the density of a floater, as described in more detail with reference to FIG. 5.

In the embodiments, background 72 has an alignment point and an alignment axis used to facilitate aligning the eye with system 10. The alignment point may be any suitable point of background 72 (e.g., near or at the center of background 72), and the alignment axis may be perpendicular to the plane of display device 20 and through the alignment point. As shown in the examples of FIGS. 2 and 3, the center 74 of background 72 is the alignment point, and the central axis 76 is the alignment axis. An axis of the eye (e.g., visual axis) may be substantially aligned with the alignment axis, e.g., central axis 76, when the eye is looking straight at the alignment point (e.g., the center 74) of background 72.

The fixation direction 78 generally goes from the point where the alignment axis intersects the retina to target 70. When the eye follows the moving target 70, the axis of the eye moves relative to central axis 76 towards fixation direction 78. Fixation angle 80 between fixation direction 78 and central axis 76 has any suitable value, which may be optimized for single saccade, e.g., a value in the range of 5 to 45 degrees, such as 10 to 30 degrees.

Computer 26 provides target display input that instructs display device 20 to display and move target 70 according to a target pattern with any suitable features. For example, a pattern may move the target to a first position, maintain the target at the first position for a dwell period (or dwell time), and after the dwell period, move the target to a second position. The dwell period is typically long enough for floater to settle at resting position, e.g., 0.5 to 5 seconds, such as 1 to 2 seconds.

As another example, a pattern may move target 70 outwards to a point away from center 74 closer (e.g., within 0.5 to 2 centimeters) to the edge of background 72 and then move target 20 towards center 74. Target 70 may stop one or more times between the outer point and center 74 to sample at different fixation angles. The sampling may be performed in any suitable manner, e.g., at steps of equal distance or varying distances, where the distance is between 1 and 10 degrees, e.g., sampling at every 5 degrees, i.e., 20, 15, 10, 5 degrees. As another example, the target display input may move the target continuously through a pattern.

FIG. 5 illustrates an example of assessment application 44 that may be used in system 10, according to certain embodiments. In the example, assessment application 44 includes score calculators 80 (80a to 80e), a severity score calculator 82, and a report generator 84. In the example, score calculators include a location score calculator 80a, an occurrence score calculator 80b, a density score calculator 80c, a situational score calculator 80d, and a patient description score calculator 80e. As an overview, score calculators 80 calculate intermediate scores for factors that can contribute to the severity of a floater, and severity score calculator 82 calculates a severity score according to one or more of the intermediate scores. Report generator 84 provides a report that includes the severity score and may also include one or more of the intermediate scores. The report may be in a format compatible for the medical staff, patient, insurance company, or other suitable party.

Score calculators 80 calculate intermediate scores for factors that can contribute to the severity of a floater. Scores may be calculated in any suitable manner. For example, a score may be determined using patient input through input device 22, a description from the patient, medical records, or a combination of any of the preceding.

Location score calculator 80a determines a location score indicating the location of the floater, e.g., where the floater is most noticeable by the patient. In general, a floater that blocks the macular causes more visual disturbance than a floater at a peripheral location. Accordingly, a floater location closer to or at the eye axis (i.e., in the central field of vision) may yield a severity score indicating greater severity, and a floater location farther away from the eye axis may yield a severity score indicating lesser severity. The location of the floater may be determined in any suitable manner. For example, the patient may describe the location of the floater in their visual field using input device 22 by, e.g., indicating whether the floater in the central field of vision or eccentric to foveal fixation, which calculator 80a can use to determine the location score.

Occurrence score calculator 80b determines an occurrence score in response to the number of times the patient indicates seeing the floater. The occurrence score may be calculated in any suitable manner. Generally, the more a patient sees the floater, the more severe the floater is. Accordingly, a greater number of times the patient indicates seeing the floater corresponds to greater severity, and a lesser number of times corresponds to lesser severity.

Density score calculator 80c determines a density score indicating the density of the floater in any suitable manner. In certain embodiments, computer 26 provides display input to adjust (e.g., increase or decrease, stepwise or continuously) the intensity of background 72, records the patient input in response to the adjusted intensity, and determines a density score according to the recorded patient input. For example, a static perimetry approach could be used where the background intensity is progressively increased until the patient sees a floater. If the patient sees the floater at a lower intensity, then the floater may have a higher density. If the patient does not see the floater until a higher intensity, then the floater may have a lower density. The relationship between intensity and density values may be determined from previous tests where patients detect floaters at specific intensity values.

In certain embodiments, computer 26 provides display input to adjust (e.g., increase or decrease, stepwise or continuously) the contrast between target 70 and background 72, records the patient input in response to the adjusted contrast, and determines a density score according to the recorded patient input. The contrast may be adjusted by changing the intensity, color, greyscale, and/or other visual feature of target 70 and/or background 72, e.g., background 72 remains constant while target 70 changes. In an example, the contrast may be progressively increased until the patient sees a floater. If the patient sees the floater at a lower contrast, then the floater may have a higher density. If the patient does not see the floater until a higher contrast, then the floater may have a lower density. The relationship between contrast and density values may be determined from previous tests where patients detect floaters at specific contrast values.

Situational score calculator 80d determines a situational score that indicates one or more factors relevant to the patient's situation, such as the living environment. For example, in certain situations the location of floater in one direction or another (e.g., left versus right or higher versus lower) may be relevant to the patient's situation. Such as, in countries where drivers drive on the right-hand side of the road, a floater on the left side of the field of vision may impair driver safety more than a floater on the right side. Patient description score calculator 80e may receive a description of the floater from the patient. The patient description may include a factor (e.g., occurrence, location, size, or other factor of the floater) that affects the severity of the floater.

Severity score calculator 82 calculates the severity score using one or more of the intermediate scores in any suitable manner. For example, severity score S may be a mathematical function f of intermediate scores pᵢ, e.g., S=f(p₁, . . . , pᵢ, . . . , pₙ), n=1, 2, 3, . . . , such as S=w₁p₁+ . . . +wᵢpᵢ+ . . . +wₙpₙ, where wᵢ represents any suitable weighting for intermediate score pᵢ. An intermediate score with a greater impact on the severity of a floater may be given a higher weighting. For example, the location score may be given a higher weight that the situational score. Report generator 84 provides a report that includes the severity score and may also include one or more intermediate scores. The report may be an output in any suitable manner, e.g., as an electronic medical record (EMR), printed report, electronic display, or other manner that communicates the severity score.

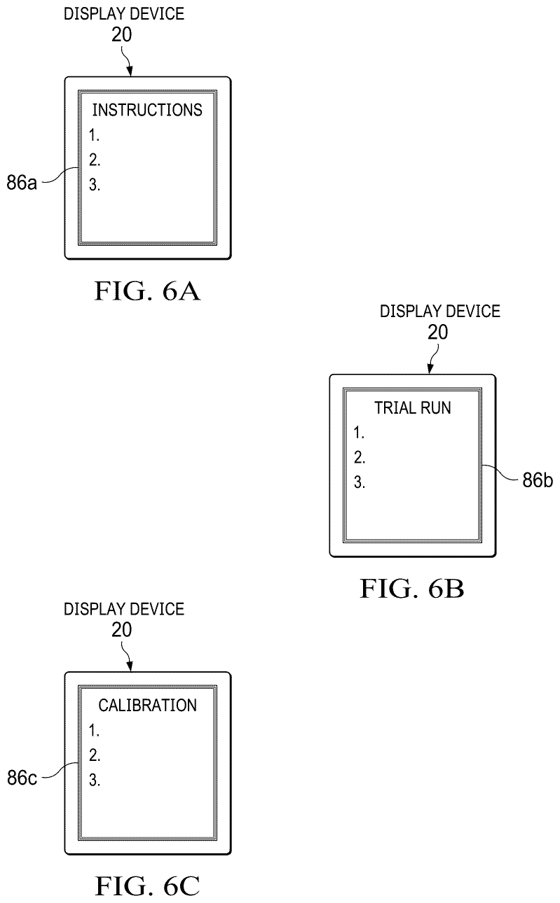

FIGS. 6A to 6C illustrate examples of graphical user interfaces (GUIs) 86 (86a to 86c) that system 10 can display to provide instructions to a patient, according to certain embodiments. In certain examples, computer 26 provides display input to display device 20 to yield the GUI 86, e.g., test instruction display input yields a test instructions GUI, trial run display input yields a trial run instructions GUI, and calibration display input yields a calibration instructions GUI. In other examples, computer 26 provides digital speech input to a speaker device to yield spoken instructions. In certain situations, the standardization of patient instructions improves efficiency and safety.

FIG. 6A illustrates an example GUI 86a that includes test instructions. The test instructions describe the floater assessment steps to the patient. The instructions may instruct the patient provide input to input device 22 when a floater is detected and may describe how the patient can provide input into input device 22, e.g., pressing a button. Example instructions may include: 1. Fixate on the target, which will move. 2. Follow the target with your gaze. 3. Press the button when you see a floater.

FIG. 6B illustrates an example GUI 86b that includes trial run instructions. The trial run instructions may explain that a trial run will be conducted to familiarize the patient with the steps of the floater assessment test before the actual test is performed. Example instructions may include: 1. We will conduct a trial run. The results will not be included in the report. 2. Fixate on the target, which will move. 3. Follow the target with your gaze. 4. Press the button when you see a floater.

FIG. 6C illustrates an example GUI 86c that includes calibration instructions. In certain embodiments, computer 26 performs a calibration procedure to gather information on how a patient reacts to a simulated floater displayed on display device 20 and uses the information to calibrate the floater assessment test. The simulated floater may be a computer graphic that mimics the appearance of a real eye floater and may have any suitable characteristic, e.g., size, shape, location, color, and/or intensity.

Computer 26 may calibrate the test in any suitable manner. In certain embodiments, the calibration procedure determines the patient's reaction time between displaying the simulated floater and receiving patient input indicating floater detection and uses the reaction time to determine the dwell period for the target. In certain embodiments, the calibration procedure displays simulated floaters with different characteristics, records patient input in response to the floaters, and compares the patient response to the simulated floaters with that of real floaters. In the embodiments, the calibration procedure may identify floater characteristics that the patient detects (or more quickly detects) as characteristics that more severely affect the patient and then give those characteristics a greater weight when calculating the severity score. For example, a patient may detect darker floaters more quickly, so the density score may be given greater weight.

The calibration instructions may include an instruction to provide input to the input device when a simulated floater is detected. Example instructions may include: 1. We will conduct a calibration procedure. 2. Fixate on the target, which will move. 3. Follow the target with your gaze. 4. A simulated floater will be displayed. 3. Press the button when you see the simulated floater.

FIGS. 7A and 7B illustrate examples of the packaging of floater assessment system 10, according to certain embodiments. FIG. 7A shows system 10 packaged as a desktop device. The desktop device may be designed to be placed on a table where a seated patient can be properly aligned with system 10. The device may be easily cleaned between use. FIG. 7B shows system 10 packaged as a near-to-eye (NTE) display device, such as goggles. The NTE display device is typically compact with a small footprint.

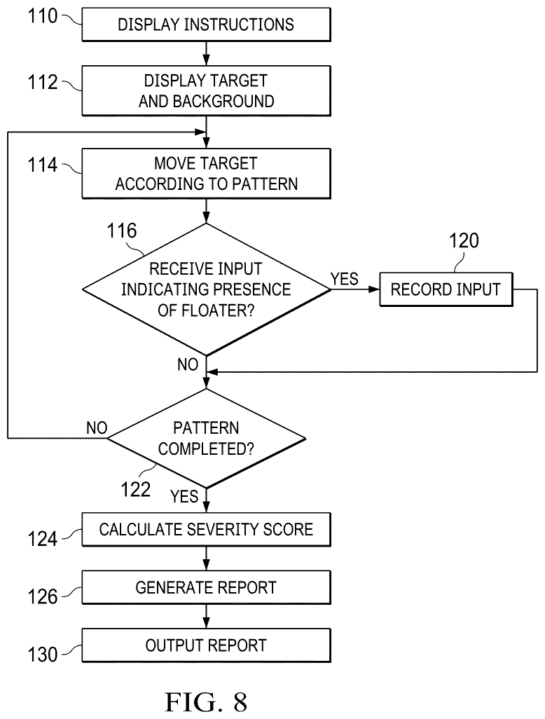

FIG. 8 illustrates an example of a method for assessing a floater in an eye of a patient that may be performed by system 10 according to certain embodiments. Display device 20 displays instructions at step 110. In an example, the instructions are test instructions that instruct the patient to fixate on a target and follow the target as it moves and to provide input to an input device (e.g., click the button on the input device) when the patient detects a floater. Display device 20 displays target 70 and background 72 at step 112. Target 70 may be any suitable size or shape, such as a dot or a cross. Computer 26 instructs display device 20 to move target 70 according to a pattern at step 114. As the patient follows the target, a floater may become more visible to the patient.

Computer 26 may receive patient input indicating that the patient sees a floater at step 116. If patient input is received, the method proceeds to step 120, where computer 26 records the input, and the method proceeds to step 122. If patient input is not received, the method proceeds directly to step 122. At step 122, the pattern may be completed. If the pattern has not been completed, the method returns to step 144, where computer 26 instructs display device 20 to move target 70 according to the pattern. If the pattern has been completed, the method proceeds to step 124.

Computer 26 calculates a severity score at step 124 that indicates the severity of the floater. In an example, computer 26 calculates the severity score from one or more intermediate scores, which may include a location score, occurrence score, density score, patient description score, situational score, and/or any other score that indicates the effect a factor has on the severity of a floater. Computer 26 generates a report that includes the severity score at step 126. The report may also include the intermediate scores and may be in a format for the medical staff, patient, insurance company, or other suitable party. Computer 26 outputs the report at step 130.

A component (such as computer 26) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112 (f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. A system for assessing a floater in an eye of a patient, comprising:
a display device configured to display a target and a background, the background having an alignment point and an alignment axis, an eye axis of the eye substantially aligned with the alignment axis of the background when the eye is fixating on the alignment point of the background;
an input device configured to receive patient input from the patient; and
a computer configured to:
provide target display input to the display device to yield the target moving relative to the background according to a pattern for the eye to follow, the eye axis of the eye moving relative to the alignment axis of the background as the eye follows the moving target;
receive the patient input indicating that the patient sees the floater;
record the patient input;
calculate a severity score according to the patient input, the severity score indicating severity of the floater; and
generate a report that describes the severity score.

2. The system of claim 1, the computer configured to provide the display input to:
move the target to a first position;
maintain the target at the first position for a dwell period, the dwell period within a range of 1 to 2 seconds; and
after the dwell period, move the target to a second position.

3. The system of claim 1, the computer configured to provide the display input to:
move the target to a position that yields a fixation direction relative to the alignment axis of the background, a fixation angle between the fixation direction and the alignment axis within a range of 10 to 30 degrees.

4. The system of claim 1, the computer configured to provide the display input to perform the following for one or more iterations:
move the target in a first direction;
move the target to the alignment point;
move the target in a second direction different from the first direction; and
move the target to the alignment point.

5. The system of claim 1, the computer configured to provide the display input to:
adjust visual contrast between the target and the background in response to a request.

6. The system of claim 1, the computer configured to calculate the severity score by:
determining a location of the floater according to the patient input;
determining a location score according to the location of the floater; and
calculating the severity score in accordance with the location score, a location closer to the eye axis corresponding to a greater severity, a location farther away from the eye axis corresponding to a lesser severity.

7. The system of claim 1, the computer configured to calculate the severity score by:
determining a density score indicating a density of the floater; and
calculating the severity score in accordance with the density score.

8. The system of claim 1, the computer configured to:
provide the display input to adjust intensity of the background;
record the patient input in response to the adjusted intensity; and
determine a density score according to the recorded patient input, the density score indicating a density of the floater.

9. The system of claim 1, the computer configured to:
provide the display input to adjust contrast between the target and the background;
record the patient input in response to the adjusted contrast; and
determine a density score according to the recorded patient input, the density score indicating a density of the floater.

10. The system of claim 1, the computer configured to calculate the severity score by:
determining an occurrence score in response to a number of times the patient indicates that the patient sees the floater; and
calculating the severity score in accordance with the occurrence score, a greater number of times corresponding to a greater severity, a lesser number of times corresponding to a lesser severity.

11. The system of claim 1, the computer configured to calculate the severity score by:
detecting a reaction time between moving the target and receiving patient input provided when a floater is detected;
determining an occurrence score in according to the reaction time; and
calculating the severity score in accordance with the occurrence score, a shorter reaction time corresponding to a greater severity, a longer reaction time corresponding to a lesser severity.

12. The system of claim 1, the computer configured to calculate the severity score by:
determining a situational score that indicates one or more factors relevant to a patient situation; and
calculating the severity score in accordance with the situational score.

13. The system of claim 1, the computer configured to calculate the severity score by:
receiving a patient description from the patient, the patient description describing the floater; and
calculating the severity score in accordance with the patient description.

14. The system of claim 1, the computer configured to:
provide test instruction display input to the display device to yield test instructions, the test instructions comprising an instruction to provide patient input to the input device when a floater is detected.

15. The system of claim 1, the computer configured to:
provide calibration display input to the display device to yield calibration instructions, the calibration instructions comprising an instruction to provide patient input to the input device when a simulated floater is detected.

16. The system of claim 1, the computer configured to:
provide calibration display input to the display device to display a simulated floater;
receive patient input indicating that the patient sees the simulated floater; and
determine a reaction time between displaying the simulated floater and receiving the patient input; and
adjust a dwell period of the pattern according to the reaction time.

17. The system of claim 1, the computer configured to:
provide calibration display input to the display device to yield calibration instructions to display a plurality of simulated floaters having floater characteristics;
receive patient input indicating that the patient sees one or more simulated floaters of the plurality of simulated floaters; and
identify the characteristics of the one or more simulated floaters indicated by the patient input.

18. The system of claim 1, the computer configured to:
provide trial run display input to the display device to yield trial run instructions for conducting a trial run of a test.

19. The system of claim 1, further comprising:
an eye tracker configured to track movement of the eye to determine a fixation position of the eye.

20. The system of claim 1, wherein one of the target or the background is lighter than the other of the target or the background.

21. The system of claim 1, wherein:
the target has a first color; and
the background has a second color that is a complement of the first color.

22. The system of claim 1, wherein the input device comprises:
an actuator configured to receive movement input from the patient.

23. The system of claim 1, wherein the input device comprises:
a microphone configured to detect sound input from the patient.

24. The system of claim 1, wherein the input device comprises:
a motion detector configured to detect gesture input from the patient.
